(19) United States
(12) Plant Patent Application Publication
(10) Pub. No.: US 2009/0193558 P1
(43) Pub. Date: Jul. 30, 2009

(54) Osteospermum plant named 'Osjaliputo'

(50) ;

(75) Inventors: Theodorus Cornelis Maria van Kleinwee, Hoorn (NL); Anna M.W.P. Houbraken, Fijnaart (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 12/011,258

(22) Filed: Jan. 25, 2008

(51) Int. Cl. A01H 5/00

(52) U.S. Cl. PLT/360

(57) ABSTRACT

A new Osteospermum plant named ‘Osjaliputo’ particularly distinguished by its very dense compact habit with many branches and short peduncles, its facing up flowers which causes strong color impact and strong contrast between young white and old purple flowers.

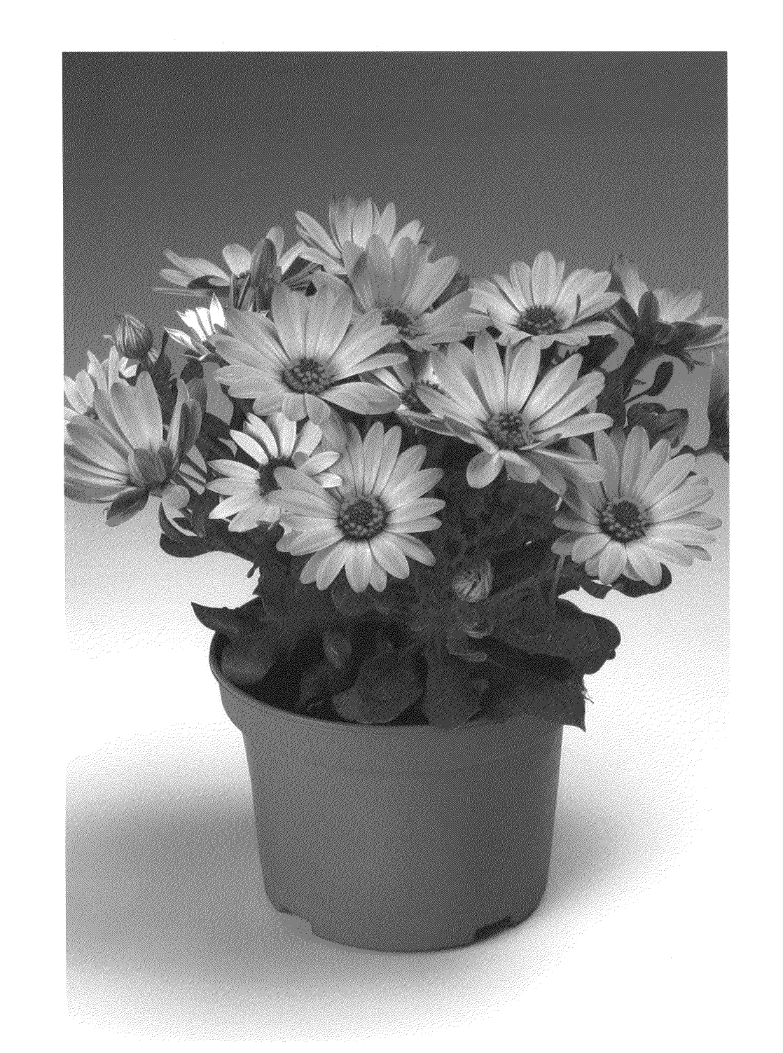

### LATIN NAME OF THE GENUS AND SPECIES OF THE PLANT CLAIMED

Osteospermum ecklonis

### VARIETAL DENOMINATION

‘Osjaliputo’

### BACKGROUND OF THE NEW PLANT

The present invention relates to a new and distinct cultivar of Osteospermum plant, botanically known as Osteospermum ecklonis.

The new Osteospermum is propagated by cuttings resulting from an open pollination of a proprietary Osteospermum ecklonis seedling selection as the female, or seed, parent identified as breeder's code ‘E0147-1’ (unpatented). Breeder's code of the new plant is ‘G0086-3.’

The new Osteospermum was discovered and selected as a single flowering plant within the progeny of the stated population grown in Enkhuizen, Netherlands, in 2002. The new Osteospermum plant has been repeatably asexually reproduced by cuttings in Enkhuizen, Netherlands since 2002. It has been found to retain its distinctive characteristics through successive propagations, and this novelty appears to be firmly fixed.

### DESCRIPTION OF THE DRAWING

This new Osteospermum plant is illustrated by the accompanying photographic drawing which shows blooms, buds and foliage of the plant in full color, the color shown being as true as can be reasonably obtained by conventional photographic procedures.

### SUMMARY OF THE INVENTION

Plants of the cultivar ‘Osjaliputo’ have not been observed under all possible environmental conditions. The phenotype may vary with variations in environment such as temperature and light intensity without, however, any variance in genotype.

The following traits have been repeatedly observed and are determined to be the unique characteristics of ‘Osjaliputo.’ These characteristics in combination distinguish ‘Osjaliputo’ as a new and distinct cultivar:
1. Dense, compact plant habit
2. Very strong branching
3. Short peduncles
4. Facing up flowers
5. Strong contrast between flower color of young white and old purple flowers

In side-by-side comparisons conducted in Enkhuizen, Netherlands, plants of the new Osteospermum differed from plants of the female parent selection in the following characteristics:
1. Plants of the new Osteospermum have a less upright and more branched growth habit than plants of the female parent.
2. Plants of the new Osteospermum have shorter peduncles than plants of the female parent.

The difference of plants of the new Osteospermum from plants of the male parent is unknown, as the male parent is unknown.

TABLE 1
DIFFERENCES BETWEEN THE NEW CULTIVAR ‘OSJALIPUTO’ AND THREE
SIMILAR CULTIVARS

| | | ‘OSJOTIS’ | | |
|---|---|---|---|---|
| | | (U.S. Plant | | ‘OSJAMLIPUR’ |
| | | Pat. No. | ‘OSECOLIPU’ | (U.S. Plant Pat. |
| | ‘OSJALIPUTO’ | 15,577) | (Not patented) | No. 17,410) |
| Plant height | 25 cm | 20-23 cm | 28 cm | 23 cm |
| Plant width | 30-35 cm | 30 cm | 50-60 cm | 32 cm |
| Flower color | old N74C | old 75A | old N78C | old N74C |
| | young 76B | young N74C | young N78C | young 76C |
| Number of | 16 | 10 | 14 | 10 |
| branches | | | | |
| Peduncle | 10-15 mm | 20-40 mm | 30 mm | 20-30 mm |
| length | | | | |
| Flower | 55-60 mm | 50-70 mm | 50-60 mm | 50 mm |
| diameter | | | | |

### BOTANICAL DESCRIPTION OF THE PLANT

The following detailed descriptions set forth the distinctive characteristics of this new Osteospermum plant The data which define these characteristics were collected from asexual reproductions carried out in Enkhuizen, Netherlands. The plant history was taken on 25 week old plants. The instant plant was grown indoors in a 10.5 cm container and transplanted into the open field in week 20.

Color references are primarily to the R.H.S. Colour Chart of The Royal Horticultural Society of London, edition 2001.
The plant:
   Classification.—Botanical: Osteospermum ecklonis.
Parentage:
   Female parent.—Proprietary seedling selection ‘E0147-1,’ not patented.
   Male parent.—Female is open pollinated so male is unknown.
Plant description:
   Growth habit.—Round, mounding.
   Plant height.—25 cm.
   Spreading area of plant.—30-35 cm.
   Strength.—Strong growth.
   Branching character.—Freely branching, 16 lateral branches without pinching.
   Blooming period.—From May until October.
The stem:
   Length.—22cm.
   Diameter.—4 mm.
   Internode length.—1 cm.
   Texture.—Glabrous.
   Color.—RHS 144A.
   Anthocyanin.—Slightly present.
The foliage:
   Arrangement.—Single leaves, alternate.
   Shape.—Elliptic.
   Apex.—Broadly acute.
   Base.—Attenuate.
   Margin.—Irregular serration.
   Number of leaves per lateral branch.—40.
   Texture.—Glabrous, leathery.
   Color upper side.—RHS 137C.
   Color lower side.—RHS 146B.
   Length.—Young 18-20 mm, mature 40-60 mm.
   Width.—Young 6-7 mm, mature 20-25 mm.
   Petiole.—Absent.
The inflorescence:
   Number of inflorescences per mature plant.—80-90.
   Inflorescence bud.—Form: Ovoid Length: 10mm Width: 7 mm Color: RHS 143C.
   Inflorescence lastingness on the plant.—Around 5 days depending on the temperature.
   Inflorescence diameter.—55-60 mm.
   Inflorescence depth.—15 mm.
   Disc diameter.—12 mm.
   Fragrance.—Absent.
   Disc florets.—Shape: Tubular, elongated Number of disc florets per inflorescence: 80 Length: 8 mm Width: 1 mm Apex: Five pointed Color: RHS 93C.
   Ray florets.—Shape: Capitulum with two series of ray florets Number of ray florets per inflorescence: 22 Color upper surface: Margin N74C-N74D, Middle 69B, base 78D Color lower surface: N77B Length: 24-27 mm Width: 5-9 mm Apex: Rounded Base: Acute Margin: Entire Texture: Satiny.
   Peduncles.—Length: 15 mm Texture: Sturdy; tiny hairs Color: RHS 144A Diameter: 1.5 mm Shape: Round.
   Phyllaries.—Texture: Rough; tiny hairs Number of phyllaries: 15-19 Color: RHS 143A; apex RHS 144B Length: 10mm Margin: Entire Width: 1mm Apex: Acute Shape: Ligulate.
Reproductive organs:
   Androecium.—Presence: Present on disc florets only Shape: Oblong, five stamens fused to one Length: 3 mm Width: 1 mm Pollen color: RHS 25A Total number of anthers: 80.
   Gynoecium.—Presence: Present on both ray and disc florets Pistil number: One pistil per ray or disc floret Pistil length: 4 mm Stigma color: RHS 77A Stigma shape: Bipartite.
Seed development: Seed development has been observed on plants of the new Osteospermum.
   Amount of seeds produced.—Moderate.
   Seed length.—6 mm.
   Seed diameter.—3 mm.
   Seed color.—RHS 200A.
   Seed shape.—Elliptic.
Disease and pest resistance: No disease/pest resistance has been observed to date.

What is claimed is:

1. A new and distinct variety of Osteospermum plant named ‘Osjaliputo,’ as substantially illustrated and described herein.
